(19) United States
(12) Patent Application Publication
(10) Pub. No.: US 2008/0097602 A1
(43) Pub. Date: Apr. 24, 2008

(54) Otologic Prostheses with Compressive Ossicular Engagement by a Superelastic Structure and Method of Implanting the Same

(76) Inventors: Robert Brosnahan, Germantown, TN (US); Richard W. Mendius, Collierville, TN (US)

Correspondence Address: GORDON & JACOBSON, P.C., 60 LONG RIDGE ROAD, SUITE 407, STAMFORD, CT, 06902, US

(21) Appl. No.: 11/551,839

(22) Filed: Oct. 23, 2006

(51) Int. Cl. A61F 2/18

(52) U.S. Cl. 623/10

(57) ABSTRACT

An ossicular prosthesis has a shaft and a superelastic engagement structure coupled to the shaft. The engagement structure at least partially defines an opening and is deformable to widen the opening to permit a portion of an ossicle to be received therein. When the engagement structure is deformed to receive the portion of the ossicle, the stress in the engagement structure remains substantially constant throughout a majority of the deformation. The load required to deform the engagement structure into an open position is relatively small, facilitating the procedure as well as reducing the potential for damage to the intact ossicle.

### BACKGROUND OF THE INVENTION

1. Field of the Invention

This invention relates broadly to prostheses. More particularly, this invention relates to otologic prostheses for replacement of the ossicles of the middle ear.

2. State of the Art

Hearing is facilitated by the tympanic membrane transforming sound in the form of acoustic sound waves within the outer ear into mechanical vibrations within the ossicular chain of bones in the middle ear. These vibrations are transmitted to the footplate of the stapes where micro (vibration) or macro motion of this structure results in compression waves within the fluid of the inner ear, These compression waves lead to vibrations of the cilia (hair cells) located within the cochlear where they are translated into nerve impulses. These nerve impulses are sent to the brain via the cochlear nerve and are interpreted in the brain as sound.

Hearing efficiency can be lost to erosion of the ossicular bones: malleus, incus, and stapes. These bones can be completely replaced by a prosthesis (total ossicular replacement prosthesis, or TORP), or various combinations of these bones can be replaced for example only the malleus and incus can be replaced (by a partial ossicular replacement prosthesis, or PORP, that assumes the presence of an intact stapes), or just the stapes can be replaced. Several types of stapedial replacements have been designed, including bucket-handle and piston designs. In a piston prosthesis, a crook-like bight is provided for placement over the incus or malleus and a shaft extends from the bight to or in some cases through the footplate.

Piston prostheses are most commonly manufactured from metals or an assembly of a metal and a polymer. The metallic portion of a traditional piston is usually manufactured from stainless steel or titanium. By way of example, a conventional piston that is adapted to be attached to the incus generally includes a bight opening which is substantially larger than the diameter of the incus. The prosthesis is attached to the incus by positioning the bight over the long process of the incus and then plastically deforming the bight to capture the incus and hold it in sufficiently close conformity to the incus for stability and vibrational transfer between the incus and the oval window. However, it may be difficult to crimp the bight in the small confines of the surgical area. Also, it is necessary to ensure that the bight is crimped evenly, but not too tightly, about the incus. Otherwise, pressure necrosis can occur to the ossicle.

U.S. Pat. Nos. 5,935,167 and 6,830,587 to a Wengen et al. describes piston prostheses made from titanium that do not require crimping. In each, a clip is defined at an upper end of the shaft of the prosthesis. The clip includes an integrated hinge-like clamp that extends outward from the clip defining two breaks about the circumference of the clamp: at the hinge-like clamp and an opposite opening for receiving the ossicle. With sufficient force, the clamp can be elastically deformed to force a portion of an ossicle, e.g., the long process of the incus or the malleus handle, between upper and lower portions of the clip where the ossicle is retained. However, pushing the ossicle through the opening of the clip requires a significant force to spread it apart. It is possible that this force could damage the ossicle. In addition, the ossicular portion is loaded by the clip at only upper and lower diametrically opposed areas, with such localized and high loading potentially leading to instability and necrosis.

The SMart™ stapes piston prosthesis from Gyrus ENT of Bartlett, Tenn., and generally described in U.S. Pat. Nos. 6,197,060 and 6,554,861 to Knox, provides a different approach that uses a shape memory alloy, for example Nitinol. This piston has the appearance of a conventional piston with its bight formed in a closed configuration. At the time of the stapedectomy, the bight is deformed into an opened configuration prior to implantation. The open bight is placed over the long process of the incus and localized heat is then applied to the bight to cause the bight to reshape into a closed configuration in accord with the shape memory material's ability to recover its as-formed original shape through heating at a phase transformation temperature. The force applied by the bight to the ossicle can be of such magnitude so as to cause long term discomfort to the patient and/or pressure induced necrosis of the bone. Furthermore, tissue structures in the vicinity of the prosthesis can be damaged as a result of the excessive application of heat to activate re-shaping of the bight. Moreover, it is possible that the heat-activated closing of the bight will result in an incomplete coupling necessary for load transmission, still requiring traditional crimping methods to complete the procedure.

### SUMMARY OF THE INVENTION

It is therefore an object of the invention to provide an ossicular prosthesis that requires fewer steps to apply to the appropriate ossicle.

It is another object of the invention to provide an ossicular prosthesis that will always couple in close conformity to an ossicle without necessitating any crimping.

It is a further object of the invention to provide an ossicular prosthesis that evenly distributes load transfer about the ossicle to which it is coupled, and will not lead to necrosis.

It is also an object of the invention to provide an ossicular prosthesis that requires minimal load to apply the prosthesis to an ossicle.

In accord with these objects, which will be discussed in detail below, an ossicular prosthesis is provided. In accord with one embodiment of the invention, the prosthesis has a shaft with a lower portion for placement at the oval window and an upper portion coupled to a curved bight adapted to compressively engage an ossicular portion such as the long process of the incus or the malleus handle. In accord with the invention, the bight is made from a superelastic metal alloy. The bight is preferably curved through at least 180° for engaging about the portion of the ossicle and at least partially defines an opening. The bight is deformable to widen the opening to permit the portion of the ossicle to be received therein. When the bight is deformed to receive the portion of the ossicle, after an initial linear loading, the stress in the bight remains substantially constant throughout its deformation, as the bight is loaded with non-linear behavior in accord with a property of the superelastic metal alloy. In accord with a preferred aspect of the invention, a handle is provided on the bight at a location displaced from the shaft and directed outward from the bight for handling the prosthesis with instrumentation.

In view of the superelastic property of the bight, the load required to deform the bight into an open position is relatively small. This reduces potential damage to the intact ossicle as well as facilitates the procedure. In addition, the bight always springs back over the ossicle, seldom requiring any secondary crimping. Moreover, the circular configuration of the bight loads the ossicle relatively evenly about its circumference with a large contact area between the bight and the ossicle, rather than at diametric locations as is done in some prior art devices. Furthermore, as the superelastic behavior is inherent in the material, the bight has a low profile that hugs the ossicle and reduces interferences and potential protrusion through the tympanic membrane.

Additional objects and advantages of the invention will become apparent to those skilled in the art upon reference to the detailed description taken in conjunction with the provided figures.

### BRIEF DESCRIPTION OF THE DRAWINGS

FIG. 1 is a side elevation of a stapedial piston prosthesis according to a first embodiment of the invention.

FIG. 2 is a graph showing the superelastic stress-strain curve of superelastic nickel titanium alloy.

FIG. 3 is a side elevation of a stapedial piston prosthesis according to a second embodiment of the invention.

FIG. 4 is a side elevation of a stapedial piston prosthesis according to a third embodiment of the invention.

FIG. 5 is a view of the stapedial piston prosthesis of the third embodiment of the invention, rotated 90° relative to FIG. 4.

FIG. 6 is a perspective view of a tympanoplasty partial ossicular replacement prosthesis (PORP).

FIG. 7 is a perspective view of an incudo-stapedial prosthesis according to the invention.

### DETAILED DESCRIPTION OF THE PREFERRED EMBODIMENTS

Turning now to FIG. 1, an ossicular prosthesis 10 according to the invention is shown. The embodiment of the prosthesis is a stapedial piston for placement at or between the oval window and the long crux (or process) of the incus. The piston 10 includes a curved bight 12 and a shaft 14. The bight 12 extends into a substantially straight upper portion 16 of the stem 14. The upper portion 16 is coupled to (or expands into) a larger diameter lower portion 18 of the shaft 14 for placement on or through the stapes footplate.

The bight 12 preferably extends through of a curve a greater than 180°, preferably at least 240°, and more preferably at least 270°, and is open at 20 to include access for receiving a portion of the ossicle about which the bight is to be engaged. The diameter of the bight is preferably approximately the same dimension or slightly smaller than the intended ossicular portion to enable slight compression about the ossicle. The bight 12 includes a free end 22 that is angled to guide the portion of the ossicle therein as the bight is maneuvered toward the ossicle. It is appreciated that the bight could be positioned and/or configured such that the opening 20 is defined between either angled free end 22 of the bight and the upper end of the upper portion 16 of the shaft, or free end 22 and another portion of the bight distanced from the upper portion 16 of the shaft. While the bight 12 is shown as crook-shaped, it is appreciated that it may be provided with another shape capable of securely engaging the target ossicular portion with a compressive force.

The bight 12 and upper portion 16 of the shaft 14 are preferably a unitary construct made of the same material, and most preferably a nickel-titanium alloy (Nitinol) wire. The wire is preferably approximately 0.10 mm in diameter. Alternatively, the bight 12 may be made from a Nitinol wire and the upper portion 16 of the shaft 14 may be made from non-superelastic material, including a metal, metal alloy or polymer and coupled to the bight, and the two may be coupled together, e.g., with adhesive, a metallurgical, or a mechanical coupling. It is also possible that bight 12 and the upper portion 16 may be manufactured from Nitinol wire and is coupled to a metallic or polymeric lower portion 18 via and adhesive, metallurgical or a mechanical coupling.

In accord with a preferred embodiment of the invention, the Nitinol wire of the bight 12 and upper portion 16 of the shaft 14 has super-elastic behavior. Referring to FIG. 2, as the prosthesis 10 is applied to an ossicle and the bight 12 is forced open, thereby increasing the strain in the Nitinol wire, after an initial short period of linear stress, the stress in the bight 12 remains substantially constant across a loading plateau. Such behavior is termed non-linear superelasticity. This superelastic behavior is observed at a temperature above the austenite finish temperature of Nitinol, and arises from the stress-induced martensitic transformation on loading and spontaneous reversion of the transformation upon unloading. Thus, as also shown in FIG. 2, upon unloading the stress also remains substantially constant across an unloading plateau. This behavior is distinct from the shape memory behavior of Nitinol which is an ability to recover an original shape upon heating through a phase transformation temperature. The production of Nitinol with superelastic or shape memory behavior is known in the art.

A handle 24 is preferably coupled to the bight 12 and preferably extends radially outward from a center of the bight. The handle 24 is displaced from the shaft 12 for handling the prosthesis with instrumentation. In addition, the handle 24 is also preferably displaced on an opposite side of the opening 20 relative to the axis A, to allow the handle 24 to operate as a lever to bend the bight into an open configuration, if desired.

The lower portion 18 of the shaft may be made from metal, such as titanium, titanium alloy, or stainless steel, or a polymer such as a polytetrafluoroethylene, and is larger in diameter than the wire shaft 16. The larger diameter is preferred, if not necessary, for implanting at the target anatomy and permitting the prosthesis to cause the required movement at the oval window to restore hearing. The overall length of the shaft 12 is generally between 3.5-7 mm so that the shaft fits the anatomy between the incus or the malleus and the oval window.

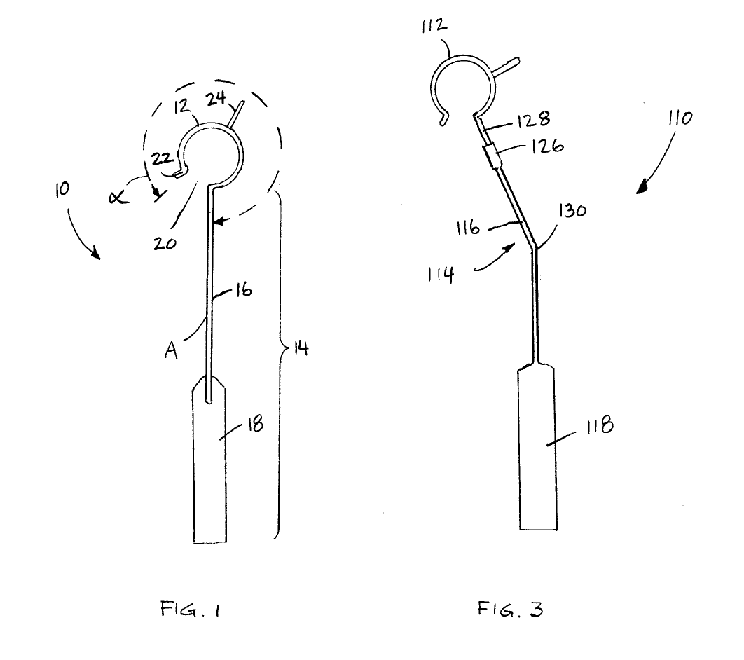

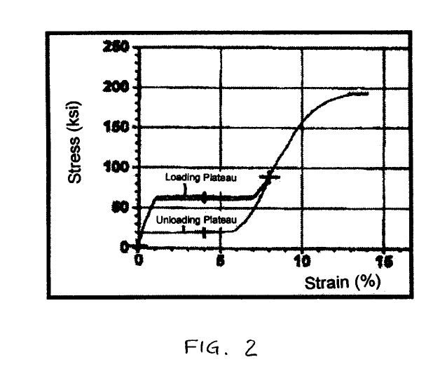

Turning now to FIG. 3, a stapedial piston 110 having a bight 112 similar to bight 12 is shown. The shaft 114 of the piston 110 is preferably slightly longer (e.g., total length up to 5-7 mm). In addition, substantially the entirety of the shaft 114 is made from a non-superelastic material, such as titanium, stainless steel, or non-superelastic nickel-titanium alloy. This allows the shaft 114 to be plastically deformable to define an angled bend, e.g., at location 130, by the surgeon or the manufacturer such that the prosthesis is extendable between the oval window and the malleus handle. The shaft 114 is preferably constructed of a common material and unitary construct from larger diameter lower portion 118 to the stepped down upper portion 116. The upper portion 116 defines an enlarged upper mount 126 for receiving an extension 128 of the bight 112. The extension 128 of the bight 112 is preferably heat melted into the mount 126, although other coupling means can be used. Alternatively, the lower portion 118 may be separately formed from upper portion 116, with the lower portion formed from the same or a different metal, metal alloy or a polymer from the upper portion.

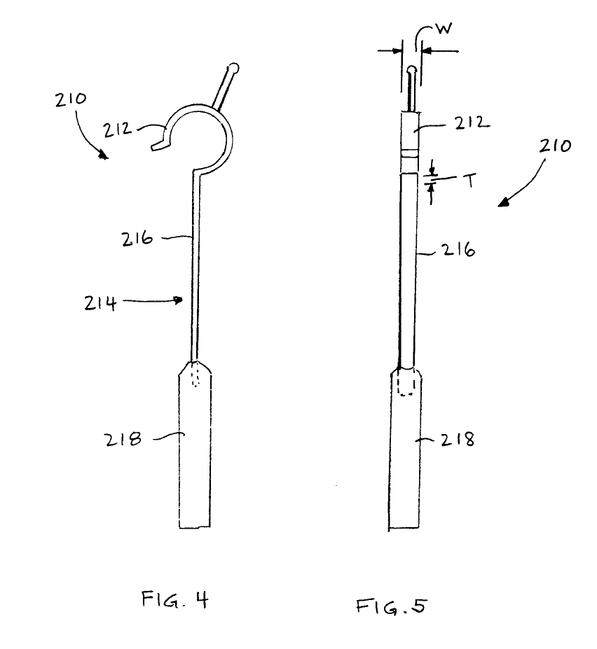

Turning now to FIGS. 4 and 5, a stapedial piston 210 substantially similar to piston 10 is shown, with the two pistons being distinguished from each other in the structure of the bights 212, 12. Piston 210 has a bight 212 that is comprised of a superelastic Nitinol ribbon having a width W substantially greater than its thickness T, whereas bight 12 of piston 10 is a shaped wire of relatively uniform diameter. The ribbon extends from the bight 212 into the upper portion 216 of the shaft 214. The ribbon is coupled to a lower portion 218 of the shaft that may be manufactured from a non-superelastic material.

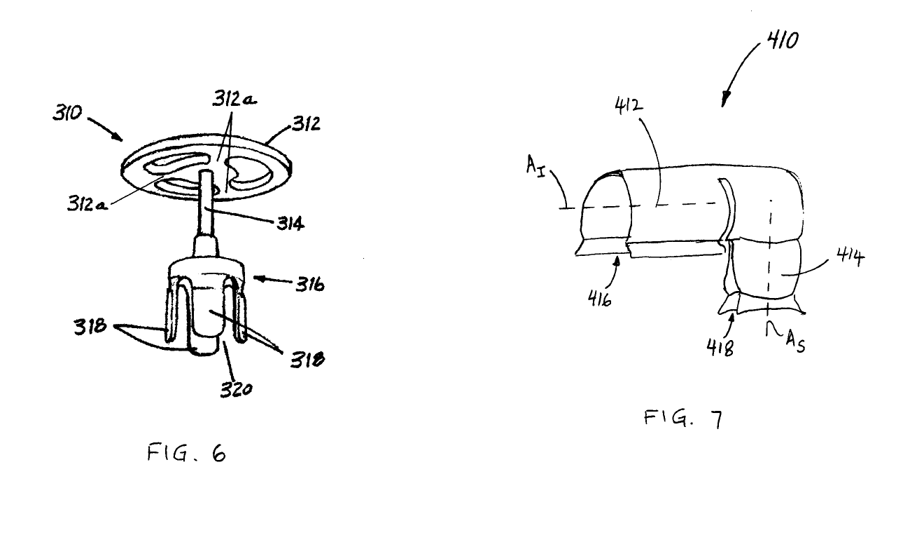

Referring now to FIG. 6, a partial prosthesis 310 for tympanoplasty is shown. Prosthesis 310 includes an oval or circular flat disc-like head 312 with spokes 312a for mating with the tympanic membrane, a clip-like shoe 316 for placement over the stapes, and a shaft 314 or other space extending means for spanning between the head and the shoe. The shaft 314 typically has a length between 1.75-3.5 mm (and preferably not exceeding 5 mm). The clip 316 is defined by at least two prongs, and preferably more than four prongs 318 adapted to stably engage the stapes under compression. The clip 316 defines an opening 320 through which the stapes can be received under compression. The prongs 318 are made from a superelastic metal alloy such as Nitinol. While the head 312 and shaft 314 may be manufactured from a superelastic alloy, it is possible for them to be made from non-superelastic materials, such as titanium, titanium alloy or stainless steel, but may be made from a polymeric material.

Turning now to FIG. 7 another embodiment of a device according to the invention is shown. The device 410 is an incudo-stapedial joint (ISJ) with two separate engagement structures 412, 414 made from superelastic nickel titanium alloy, each engagement structure being deformed when placed on incus and stapes respectively, and then engaging a separate portion of ossicle under compression. The engagement structure are provided on different axes A₁ (axis of incus) and A_{S} (axis of stapes) which are oriented transversely to each other. However, both engagement structure have openings 416, 418 at a lower end.

In each embodiment, the superelastic behavior of the bight or clip permits such engagement structure to be deformed with low load to permit entrance of an ossicular portion within the engagement structure. The engagement structure is deformable to widen the opening to permit a portion of an ossicle (e.g., long process of incus, malleus head, capitulum of the stapes) to be received therein and, in accord with the superelastic behavior of the Nitinol material, after a short period of linear stress, the stress in the engagement structure remains substantially constant during such deformation. Such period of linear stress is substantially short in duration such that the engagement structure is subject to substantially constant stress (in a non-linear relationship to the applied strain) throughout the majority of the engagement structure deformation. Then, as the ossicular portion is received in the engagement structure, the engagement structure does not forcibly snap on the ossicle, but rather will deform with low but constant compressive force thereabout it. In addition, the superelastic behavior also substantially evenly distributes force about the ossicle, rather than concentrate the loading force. Further, the engagement structure will always attempt to recover to assume its original shape which conforms to the ossicle for enhanced stability and security and load transfer. In addition, the prosthesis maintains a low profile, reducing the potential for interference within other ossicles, or the potential for protrusion through the tympanic membrane.

There have been described and illustrated herein several embodiments of a ossicular prosthesis, and particularly a prostheses for stapedioplasty and tympanoplasty and a method of implanting the same. While particular embodiments of the invention have been described, it is not intended that the invention be limited thereto, as it is intended that the invention be as broad in scope as the art will allow and that the specification be read likewise. Moreover, while specific embodiments of prostheses have been disclosed, it is appreciated that other embodiments for prostheses for replacements of one or more ossicles or the joints therebetween can be provided, with such embodiments having an engagement structure that partially surrounds and engages a portion of an ossicle under compression. It will therefore be appreciated by those skilled in the art that yet other modifications could be made to the provided invention without deviating from its spirit and scope as claimed.

1-10. (canceled)

11. An ossicular prosthesis, comprising:
a flat head portion for mating with the tympanic membrane;
a superelastic metal alloy engagement structure for providing a compressive force about a portion of an ossicle; and
a shaft extending between said head portion and said engagement structure and having a length not exceeding approximately 5 mm,
wherein said engagement structure defines an opening and is deformable to widen the opening to permit the portion of the ossicle to be received therein, and when the engagement structure is deformed to receive the portion of the ossicle the stress in the engagement structure, after a short duration of linear elastic behavior, remains substantially constant.

12. An ossicular prosthesis, comprising:
a first superelastic metal alloy engagement structure for providing a compressive force about a portion of an ossicle; and
a second superelastic metal alloy engagement structure for providing a compressive force about a portion of an ossicle,
wherein each respective engagement structure defines an opening and is deformable to widen the opening to permit the respective portion of the ossicle to be received therein, and when the respective engagement structure is deformed to receive the portion of the ossicle the stress in the respective engagement structure, after a short duration of linear elastic behavior, remains substantially constant.

13. An ossicular prosthesis according to claim 12, wherein:
said first and second engagement structures are provided along different axes of said prosthesis.

14. An ossicular prosthesis according to claim 13, wherein:
openings into both said first and second engagement structure are from a common direction.

15. A method of implanting an ossicular prosthesis, comprising:
a) providing an ossicular prosthesis having a first engagement structure defining a first opening through which a portion of a first ossicle can be received therein and a second engagement structure defining a second opening through which a portion of a second ossicle can be received therein, the first and second engagement structures adapted to apply a compressive force to restrain the respective first and second ossicles received therein, the engagement structure made from superelastic nickel titanium alloy; and
b) implanting the ossicular prosthesis, wherein during the implantation the first engagement structure is deformed to widen the first opening to permit the portion of the first ossicle to be received therein, and the second engagement structure is deformed to widen the second opening to permit the portion of the second ossicle to be received therein, and
when the first engagement portion is deformed to receive the portion of the first ossicle, the stress in the first engagement structure remains substantially constant throughout a majority of its deformation, and after the portion of the first ossicle is received therein the first engagement portion applies a compressive force to the portion of the first ossicle, and
when the second engagement portion is deformed to receive the portion of the second ossicle, the stress in the second engagement structure remains substantially constant throughout a majority of its deformation, and after the portion of the second ossicle is received therein the second engagement portion applies a compressive force to the portion of the second ossicle.

16-17. (canceled)

18. A method according to claim 15, wherein:
said first and second engagement structures are made from superelastic nickel titanium alloy.

19. A method according to claim 15, wherein:
said first and second openings are placed over said portions of said first and second ossicles from a common direction.

20. A method according to claim 15, wherein:
said first and second engagement structures are oriented in a generally L-shape configuration.

21. A method according to claim 15, wherein:
axes extending longitudinally through said first and second engagement structures are oriented transversely to each other.

22. A method according to claim 15, wherein:
axes extending longitudinally through said first and second engagement structures are oriented perpendicularly to each other.

23. A method according to claim 15, wherein:
said implanting replaces a joint between an existing incus and stapes.
